(19) United States
(12) Patent Application Publication
(10) Pub. No.: US 2025/0064616 A1
(43) Pub. Date: Feb. 27, 2025

(54) EXPANDABLE STENT WITH IMPROVED DELIVERABILITY

(71) Applicant: BOSTON SCIENTIFIC SCIMED, INC., Maple Grove, MN (US)

(72) Inventor: Martyn G. Folan, Galway (IE)

(73) Assignee: BOSTON SCIENTIFIC SCIMED, INC., Maple Grove, MN (US)

(21) Appl. No.: 18/814,102

(22) Filed: Aug. 23, 2024

Related U.S. Application Data
(60) Provisional application No. 63/578,511, filed on Aug. 24, 2023.

(51) Int. Cl. A61F 2/966; A61F 2/848; A61F 2/90; A61F 2/95

(52) U.S. Cl. CPC A61F 2/966; A61F 2/848; A61F 2/90; A61F 2002/8483; A61F 2002/9511; A61F 2002/9665; A61F 2210/0057; A61F 2230/0019; A61F 2250/0007; A61F 2250/001

(57) ABSTRACT

A polymeric stent includes a polymeric tubular body extending from a distal region to a proximal region, the polymeric tubular body adapted to have a negative Poisson's ratio. A distal engagement feature is formed within the distal region and a proximal engagement feature is formed within the proximal region. A delivery device for the polymeric stent includes a tubular body having an outer surface adapted to accommodate the polymeric stent thereover. A distal stent engagement member is secured relative to the tubular body and is adapted to releasably engage the distal engagement feature. A proximal stent engagement member is secured relative to the tubular body and is adapted to releasably engage the proximal engagement feature. The tubular body is adapted to affect a change in a distance between the distal stent engagement member and the proximal stent engagement member, thereby changing a length of the polymeric stent.

### CROSS REFERENCE TO RELATED APPLICATIONS

The present application claims the benefit of and priority to U.S. Provisional Patent Application Ser. No. 63/578,511, filed on Aug. 24, 2023, the disclosure of which is incorporated herein by reference.

### TECHNICAL FIELD

The present disclosure relates generally to methods and apparatuses for various digestive ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain the structure open. These devices are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof.

An example may be found in a polymeric stent adapted for placement within a biliary duct. The polymeric stent includes a polymeric tubular body extending from a distal region to a proximal region. The polymeric tubular body has a delivery configuration with a first diameter and a first length. The polymeric tubular body has a deployed configuration with a second diameter greater than the first diameter and a second length greater than the first length. The polymeric tubular body is adapted to increase in diameter from the first diameter towards the second diameter in response to the polymeric tubular body being stretched axially from the first length towards the second length.

Alternatively or additionally, the polymeric tubular body may include an auxetic structure.

Alternatively or additionally, the auxetic structure may include one of a rotating rectangles structure, a re-entrant structure or a chiral structure.

Another example may be found in an assembly. The assembly includes a polymeric stent and a delivery device adapted to deliver the polymeric stent. The polymeric stent includes a polymeric tubular body extending from a distal region to a proximal region, the polymeric tubular body adapted to have a negative Poisson's ratio. A distal engagement feature is formed within the distal region and a proximal engagement feature is formed within the proximal region. The delivery device includes a tubular body adapted to accommodate the polymeric stent disposed over the tubular body. A distal stent engagement member is secured relative to the tubular body, the distal stent engagement member adapted to releasably engage the distal engagement feature. A proximal stent engagement member is secured relative to the tubular body, the proximal stent engagement member adapted to releasably engage the proximal engagement feature. The tubular body is adapted to affect a change in a distance between the distal stent engagement member and the proximal stent engagement member, thereby changing a length of the polymeric stent.

Alternatively or additionally, increasing the length of the polymeric stent may result in the polymeric stent increasing in diameter.

Alternatively or additionally, subsequently decreasing the length of the polymeric stent after increasing the length of the polymeric stent may result in the polymeric stent decreasing in diameter.

Alternatively or additionally, the distal stent engagement member and the proximal engagement member may each adapted to allow the polymeric stent to disengage from the distal stent engagement member and the proximal engagement member once the proximal stent reaches a predetermined length.

Alternatively or additionally, the distal stent engagement member may include a distal protrusion extending above the outer surface of the tubular body and the proximal stent engagement member may include a proximal protrusion extending above the outer surface of the tubular body.

Alternatively or additionally, the distal engagement feature may include a distal aperture formed within the distal region, the distal aperture adapted to releasably engage the distal stent engagement member, and the proximal engagement feature may include a proximal aperture formed within the proximal region, the proximal aperture adapted to releasably engage the proximal stent engagement member or, alternatively. The distal engagement feature may include a distal barb formed within the distal region, the distal barb adapted to releasably engage the distal stent barb member, and the proximal engagement feature may include a proximal aperture formed within the proximal region, the proximal barb adapted to releasably engage the proximal stent engagement member.

Alternatively or additionally, the tubular body may include an elongate outer member including an outer surface, and an elongate inner member, the elongate inner member including an outer surface. The distal stent engagement member may be secured to the outer surface of the elongate outer member and the proximal stent engagement member may be secured to the outer surface of the elongate inner member. The elongate inner member may be adapted to translate within the elongate outer member, thereby changing the distance between the distal stent engagement member and the proximal stent engagement member.

Alternatively or additionally, the polymeric tubular body may include one of a rotating rectangles structure, a re-entrant structure or a chiral structure.

Another example may be found in a delivery device for delivering a polymeric stent that includes a polymeric tubular body adapted to grow radially in response to being stretched axially, the polymeric stent including a first engagement feature and a second engagement feature. The delivery device includes a telescoping body including an elongate outer member and an elongate inner member, the elongate inner member adapted to translate within the elongate outer member. A first stent engagement member is secured relative to the elongate outer member, the first stent engagement member adapted to releasably engage the first engagement feature. A second stent engagement member is secured relative to the elongate inner member, the second stent engagement member adapted to releasably engage the second engagement feature. Translating the elongate inner member in a first direction relative to the elongate outer member causes a distance between the first stent engagement member and the second stent engagement member to increase, thereby stretching the polymeric stent and causing the polymeric stent to increase in diameter.

Alternatively or additionally, the distal stent engagement member and the proximal engagement member may each adapted to allow the polymeric stent to disengage from the distal stent engagement member and the proximal engagement member once the proximal stent reaches a predetermined length.

Alternatively or additionally, the distal stent engagement member may include a distal protrusion extending radially away from the elongate outer member and the proximal stent engagement member may include a proximal protrusion extending radially away from the elongate inner member.

Alternatively or additionally, the elongate inner member may include a distal stop disposed at a distal end of the elongate inner member, the distal stop adapted to limit how far the elongate inner member can be withdrawn from the elongate outer member.

Another example may be found in an assembly. The assembly includes a polymeric stent and a delivery device adapted to deliver the polymeric stent. The polymeric stent includes a polymeric tubular body extending from a distal region to a proximal region, the polymeric tubular body adapted to have a negative Poisson's ratio. A distal engagement feature is formed within the distal region and a proximal engagement feature is formed within the proximal region. The delivery device includes a tubular body adapted to accommodate the polymeric stent disposed over the tubular body. A distal stent engagement member is secured relative to the tubular body, the distal stent engagement member adapted to releasably engage the distal engagement feature, and a proximal stent engagement member is secured relative to the tubular body, the proximal stent engagement member adapted to releasably engage the proximal engagement feature. The tubular body is adapted to affect a change in a distance between the distal stent engagement member and the proximal stent engagement member, thereby changing a length of the polymeric stent.

Alternatively or additionally, increasing the length of the polymeric stent may result in the polymeric stent increasing in diameter.

Alternatively or additionally, subsequently decreasing the length of the polymeric stent after increasing the length of the polymeric stent may result in the polymeric stent decreasing in diameter.

Alternatively or additionally, the distal stent engagement member and the proximal engagement member may each adapted to allow the polymeric stent to disengage from the distal stent engagement member and the proximal engagement member once the proximal stent reaches a predetermined length.

Alternatively or additionally, the distal stent engagement member may include a distal protrusion extending above the outer surface of the tubular body and the proximal stent engagement member may include a proximal protrusion extending above the outer surface of the tubular body.

Alternatively or additionally, the distal engagement feature may include a distal aperture formed within the distal region, the distal aperture adapted to releasably engage the distal stent engagement member, and the proximal engagement feature may include a proximal aperture formed within the proximal region, the proximal aperture adapted to releasably engage the proximal stent engagement member.

Alternatively or additionally, the distal engagement feature may include a distal barb formed within the distal region, the distal barb adapted to releasably engage the distal stent barb member, and the proximal engagement feature may include a proximal aperture formed within the proximal region, the proximal barb adapted to releasably engage the proximal stent engagement member.

Alternatively or additionally, the tubular body may include an elongate outer member including an outer surface, and an elongate inner member including an outer surface. The distal stent engagement member may be secured to the outer surface of the elongate outer member and the proximal stent engagement member may be secured to the outer surface of the elongate inner member.

Alternatively or additionally, the elongate inner member may be adapted to translate within the elongate outer member, thereby changing the distance between the distal stent engagement member and the proximal stent engagement member.

Alternatively or additionally, the polymeric tubular body may include a rotating rectangles auxetic structure.

Alternatively or additionally, the polymeric tubular body may include a re-entrant auxetic structure.

Alternatively or additionally, the polymeric tubular body may include a chiral auxetic structure.

Another example may be found in a delivery device for delivering a polymeric stent that includes a polymeric tubular body adapted to grow radially in response to being stretched axially, the polymeric stent including a first engagement feature and a second engagement feature. The delivery device includes a telescoping body including an elongate outer member and an elongate inner member, the elongate inner member adapted to translate within the elongate outer member. A first stent engagement member is secured relative to the elongate outer member, the first stent engagement member adapted to releasably engage the first engagement feature, and a second stent engagement member is secured relative to the elongate inner member, the second stent engagement member adapted to releasably engage the second engagement feature. Translating the elongate inner member in a first direction relative to the elongate outer member causes a distance between the first stent engagement member and the second stent engagement member to increase, thereby stretching the polymeric stent and causing the polymeric stent to increase in diameter.

Alternatively or additionally, the distal stent engagement member and the proximal engagement member may each adapted to allow the polymeric stent to disengage from the distal stent engagement member and the proximal engagement member once the proximal stent reaches a predetermined length.

Alternatively or additionally, the distal stent engagement member may include a distal protrusion extending radially away from the elongate outer member and the proximal stent engagement member may include a proximal protrusion extending radially away from the elongate inner member.

Alternatively or additionally, the elongate inner member may include a distal stop disposed at a distal end of the elongate inner member, the distal stop adapted to limit how far the elongate inner member can be withdrawn from the elongate outer member.

Alternatively or additionally, the polymeric stent may include a first barb and associated hollow as the first engagement feature and a second barb and associated hollow as the second engagement feature. The first stent engagement member may be adapted to releasably engage the first barb and associated hollow and the second stent engagement member may be adapted to releasably engage the second barb and associated hollow.

Another example may be found in a polymeric stent adapted for placement within a biliary duct. The polymeric stent includes a polymeric tubular body extending from a distal region to a proximal region. The polymeric tubular body has a delivery configuration with a first diameter and a first length. The polymeric tubular body has a deployed configuration with a second diameter greater than the first diameter and a second length greater than the first length. The polymeric tubular body is adapted to increase in diameter from the first diameter towards the second diameter in response to the polymeric tubular body being stretched axially from the first length towards the second length.

Alternatively or additionally, the polymeric tubular body may include an auxetic structure.

Alternatively or additionally, the auxetic structure may include one of a rotating rectangles auxetic structure, a re-entrant auxetic structure or a chiral auxetic structure.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative polymeric stent shown in a delivery configuration with a first length and a first diameter;

FIG. 2 is a schematic view of the example polymeric stent of FIG. 1, shown in a deployment configuration with a second length greater than the first length and a second diameter greater than the first diameter;

FIG. 3 is a schematic view of an example auxetic structure;

FIG. 4A is a schematic view of an example auxetic structure in an undeformed configuration and FIG. 4B is a schematic view of the example auxetic structure in a deformed configuration;

FIG. 5A is a schematic view of an example auxetic structure in an undeformed configuration and FIG. 5B is a schematic view of the example auxetic structure in a deformed configuration;

FIG. 6A is a schematic view of an example auxetic structure in an undeformed configuration and FIG. 6B is a schematic view of the example auxetic structure in a deformed configuration;

FIG. 7A is a schematic view of an example auxetic structure in an undeformed configuration and FIG. 7B is a schematic view of the example auxetic structure in a deformed configuration;

FIGS. 8A through 8H are schematic views of example polymeric stents;

FIGS. 9A through 9C are schematic views of an example delivery device;

FIGS. 10A and 10B together show an example implantation;

FIGS. 11A through 11C together show an example implantation; and

FIGS. 12A and 12B are schematic views of an example polymeric stent.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

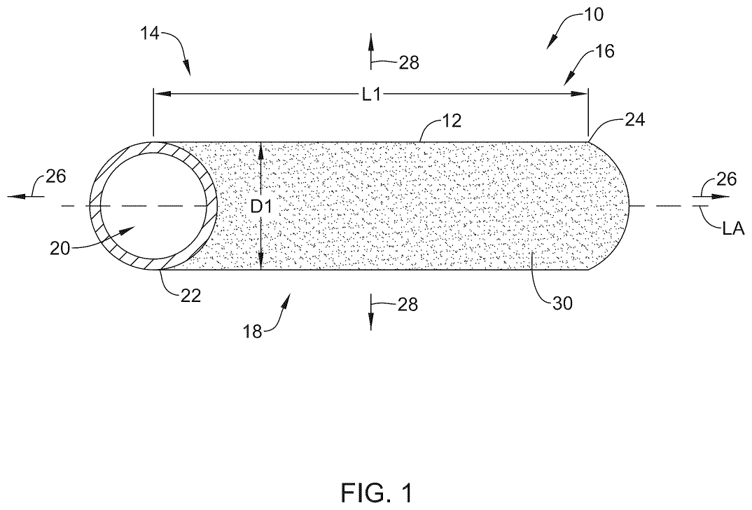

Stents are utilized in a variety of different body lumens, including the vasculature and various parts of the gastrointestinal system, for example. FIG. 1 is a schematic view of an example polymeric stent 10 that is adapted for use in body lumens in which the polymeric stent 10 will be subjected to various movement forces. The polymeric stent 10 includes a polymeric tubular body 12. While the polymeric tubular body 12 is shown as generally tubular, it is contemplated that the polymeric tubular body 12 may take any cross-sectional shape desired. The polymeric stent 10 may be considered as including a distal region 14, a proximal region 16, and an intervening intermediate region 18, recognizing that these designations are arbitrary, and depend on the orientation in which the polymeric stent 10 will ultimately be implanted within a body lumen. The polymeric stent 10 may be considered as having a constant diameter D₁ throughout, including through the distal region 14, the proximal region 16 and the intervening intermediate region 18. In some instances, the polymeric stent 10 may be considered as having a constant diameter D₁ when in a relaxed state. The polymeric stent 10 may include a lumen 20 that extends from a distal end 22 of the polymeric stent 10 to a proximal end 24 of the polymeric stent 10 to allow for the passage of food, fluids, etc. The polymeric stent 10 may be considered as having a longitudinal axis LA.

The polymeric stent 10 has a length L₁. In some instances, there may be a relationship between the diameter D₁ and the length L₁. The polymeric stent 10 shown in FIG. 1 may be considered as being in a delivery configuration. In some instances, the polymeric stent 10 may be adapted to be stretched in length by applying a tensile force in a direction indicated by arrows 26, parallel with the longitudinal axis LA. In response to the applied tensile force, and the polymeric stent 10 increasing in length, the polymeric stent 10 may also increase in diameter, increasing in a direction indicated by arrows 28. In some instances, the polymeric stent 10, or at least the polymeric tubular body 12 may be adapted to increase in diameter in response to an applied tensile force along the longitudinal axis LA. In some instances, the polymeric stent 10 and/or the polymeric tubular body 12 may be considered as having a negative Poisson's ratio. In some instances, the polymeric stent 10 and/or the polymeric tubular body 12 may be considered as being auxetic.

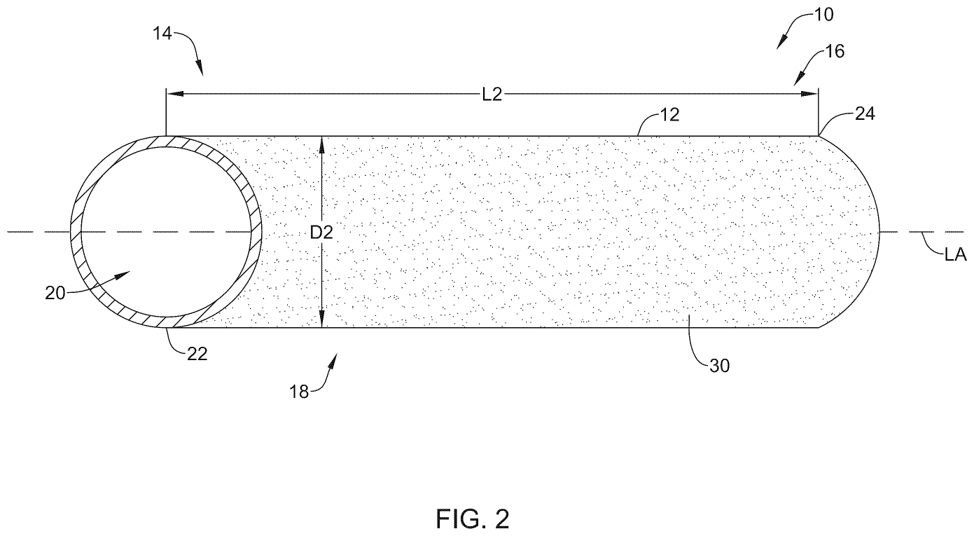

FIG. 2 shows the polymeric stent 10 in an enlarged configuration, relative to that shown in FIG. 1. In FIG. 2, the polymeric stent 10 has a length L₂ that is greater than the length L₁ shown in FIG. 1 as a result of the polymeric stent 10 being stretched axially. Moreover, the polymeric stent 10 has a diameter D₂ that is greater than the diameter D₁ shown in FIG. 1. Because the polymeric stent 10 has increased in length, from a length of L₁ to a length of L₂, the polymeric stent 10 has correspondingly increased in diameter, increasing from a diameter D₁ to a diameter D₂. This is a property of materials that are auxetic, or that demonstrate a negative Poisson's ratio. In some instances, depending on the particular auxetic construction of the polymeric stent 10, there may or may not be a linear relationship between a change in length and a corresponding change in diameter. As an example, D₂ may be about 120 percent of D₁, or about 150 percent of D₁, or perhaps about 200 percent of D₁. L₂ may be about 90 percent of L₁, or about 75 percent of L₁, or about 50 percent of L₁.

Most materials have a positive Poisson's ratio. Poisson's ratio of a material is the ratio of the lateral contractile strain to the longitudinal tensile strain for a material undergoing tension in a longitudinal direction. In other words, materials having a positive Poisson's ratio become thinner radially when stretched axially, and will become thicker radially when compressed axially. In contrast, a material having a negative Poisson's ratio, such as the material forming the polymeric tubular body 12 shown in FIG. 1, is just the opposite. A material having a negative Poisson's ratio will become thicker radially when stretched axially, and will become thinner radially when compressed axially. In some instances, materials having a negative Poisson's ratio, or auxetic materials, may operate at any scale ranging from a nano-level (molecular level) to a macroscale. A material such as a polymeric material may be made to be auxetic by modifying the material on any of a nano-scale to a macro-scale. In some instances, a polymeric material may be made to be auxetic by forming a structure using an additive process such as 3D printing, for example.

Accordingly, the polymeric stent 10, by virtue of being made from an auxetic material, can be moved from a delivery configuration as shown in FIG. 1 to a deployment configuration as shown in FIG. 2, simply by stretching the polymeric stent 10 axially. In some instances, depending on the particular nature of the auxetic material used to form the polymeric tubular body 12, the polymeric stent 10 may for example include a polymeric covering 30, indicated via a dotted pattern overlaid on the polymeric tubular body 12. In some instances, the polymeric covering 30, if included, may be adapted to stretch axially and radially as the polymeric tubular body 12 under the polymeric covering 30 increases in length and diameter.

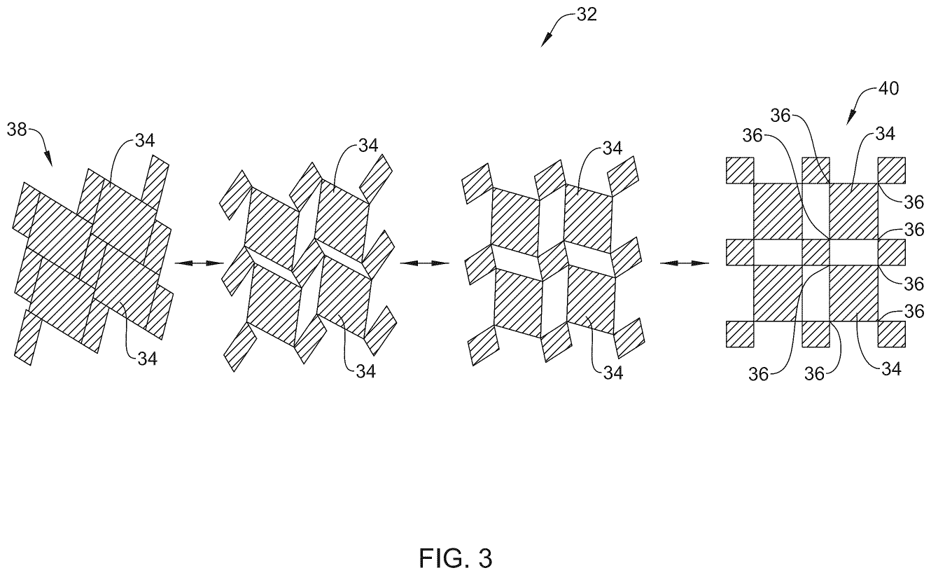

FIG. 3 is a schematic view of an example auxetic structure 32 that includes a number of rectilinear shapes 34 that are hinged together at their vertices. It will be appreciated that each rectilinear shape 34 has a total of four vertices 36 and thus each rectilinear shape 34 may be hingedly connected to a total of four other rectilinear shapes 34. Each of the rectilinear shapes 34 may have equal length sides, and thus may be squares. Each of the rectilinear shapes 34 may have two sides that are equal in length, and two other sides that are equal in length, but not equal in length to the first two sides, and thus may be rectangles. In FIG. 3, a configuration 38, on the left side of the Figure, may represent a reduced length (and diameter) configuration, and a configuration 40, on a right side of the Figure, may represent an increased length (and diameter) configuration as a result of the configuration 38 being stretched axially. The auxetic structure 32 may be formed from any of a variety of known polymers, including polymers known in medical applications. Providing the polymeric tubular body 12 with the auxetic structure 32 will permit the polymeric tubular body 12 to increase in size radially in response to stretching the polymeric tubular body 12 axially.

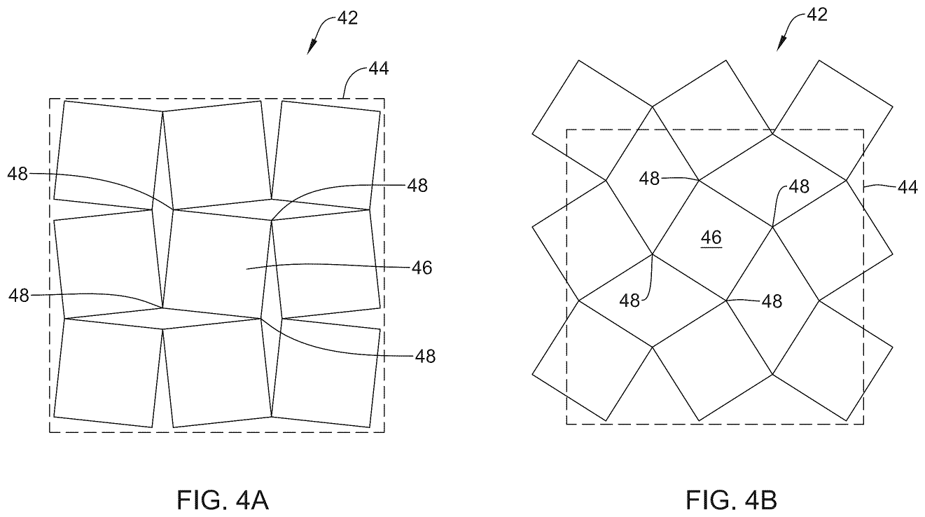

FIG. 4A is a schematic view of an example auxetic structure 42 shown in an undeformed or native configuration while FIG. 4B is a schematic view of the example auxetic structure 42 shown in a deformed configuration in which the auxetic structure 42 has increased in diameter as a result of being stretched axially. The auxetic structure 42 may be considered as being an example of a rotating rectangles auxetic structure. A difference between the undeformed configuration and the deformed configuration may be seen by comparing a dashed box 44 that in FIG. 4A, surrounds the auxetic structure 42, while in FIG. 4B, the auxetic structure 42 is wider and taller than the auxetic structure 42 as shown in FIG. 4A. The auxetic structure 42 includes a number of rectilinear shapes 46 joined together at their respective vertices 48 in a manner that allows each rectilinear shape 46 to rotate. In particular, a particular rectilinear shape 46 is connected at each of its four vertices 48 to only one other rectilinear shape 46, which allows the rectilinear shapes 46 to rotate relative to each other.

In FIG. 4A, the rectilinear shapes 46 are closer together, in a more compact arrangement. In FIG. 4B, the rectilinear shapes 46 are farther apart, in a more spread apart arrangement, and thus the auxetic structure 42 is wider and taller, which may occur as a result of the auxetic structure 42 being stretched. Each of the rectilinear shapes 46 may have equal length sides, and thus may be squares. Each of the rectilinear shapes 46 may have two sides that are equal in length, and two other sides that are equal in length, but not equal in length to the first two sides, and thus may be rectangles. The auxetic structure 42 may be formed from any of a variety of known polymers, including polymers known in medical applications. Providing the polymeric tubular body 12 with the auxetic structure 42 will permit the polymeric tubular body 12 to increase in size radially in response to stretching the polymeric tubular body 12 axially.

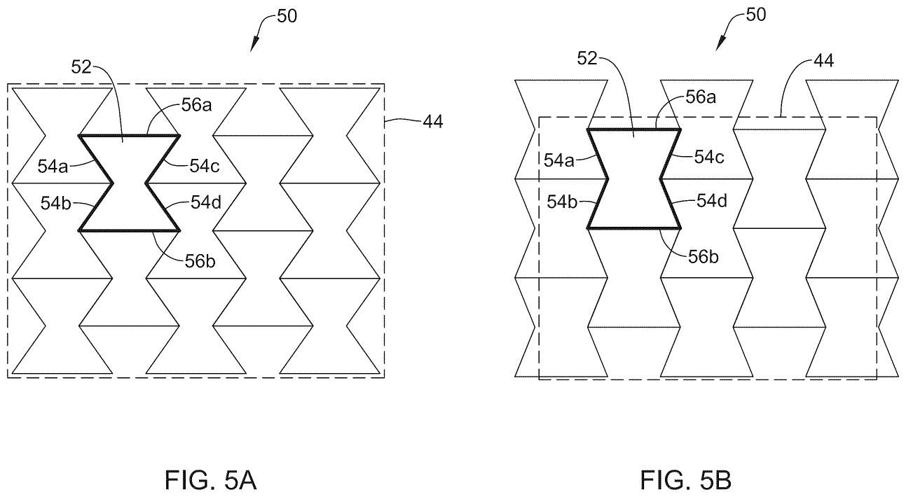

FIG. 5A is a schematic view of an example auxetic structure 50 shown in an undeformed or native configuration while FIG. 5B is a schematic view of the example auxetic structure 50 shown in a deformed configuration in which the auxetic structure 50 has increased in diameter as a result of being stretched axially. The auxetic structure 50 may be considered as being an example of a re-entrant auxetic structure. A difference between the undeformed configuration and the deformed configuration may be seen by comparing the dashed box 44 that in FIG. 5A, surrounds the auxetic structure 50, while in FIG. 5B, the auxetic structure 50 is wider and taller than the auxetic structure 50 as shown in FIG. 4A.

The auxetic structure 50 includes a number of honeycomb shapes 52, one of which is bolded in each of FIG. 5A and FIG. 5B. Each honeycomb shape 52 may be considered as being formed from a number of struts, including side struts 54a and 54b on a first side of the indicated honeycomb shape 52, and side struts 54c and 54d on an opposing second side of the indicated honeycomb shape 52. Each honeycomb shape 52 includes a top strut 56a (in the illustrated orientation) and a bottom strut 56b. When a tensile force is applied, stretching the auxetic structure 52, the angles between the struts can vary, allowing each honeycomb shape 52 to become both taller and wider. In particular, and in comparing FIG. 5A with 5B, an angle between the side struts 54a and 54b, and between the side struts 54c and 54d, has increased in FIG. 5B relative to FIG. 5A. Similarly, an angle formed between the top strut 56a and the side strut 54a, and between the top strut 56a and the side strut 54c, and between the bottom strut 56b and the side strut 54b, and between the bottom strut 56g and the side strut 54d, have each increased in FIG. 5B relative to FIG. 5A. The auxetic structure 50 may be formed from any of a variety of known polymers, including polymers known in medical applications. Providing the polymeric tubular body 12 with the auxetic structure 50 will permit the polymeric tubular body 12 to increase in size radially in response to stretching the polymeric tubular body 12 axially.

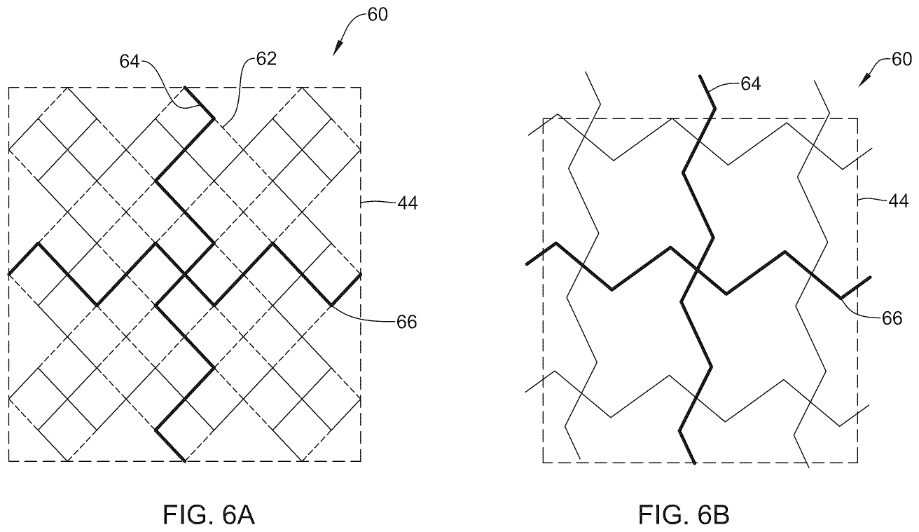

FIG. 6A is a schematic view of an example auxetic structure 60 shown in an undeformed or native configuration while FIG. 6B is a schematic view of the example auxetic structure 60 shown in a deformed configuration in which the auxetic structure 60 has increased in diameter as a result of being stretched axially. The auxetic structure 60 may be considered as being an example of a missing rib auxetic structure. A difference between the undeformed configuration and the deformed configuration may be seen by comparing a dashed box 44 that in FIG. 6A, surrounds the auxetic structure 60, while in FIG. 6B, the auxetic structure 60 is wider and taller than the auxetic structure 60 as shown in FIG. 6A. The auxetic structure 60 may be considered as missing a number of rib structures relative to a non-modified structure. In FIG. 6A, a number of rib segments 62 are shown in dashed line to show what has been removed.

As a result of the rib segments 62 that are not present, the auxetic structure 60 may be considered as including a number of vertical (in the illustrated orientation) ribs 64 and horizontal ribs 66. A representative vertical rib 64 and a representative horizontal rib 66 are highlighted. In FIG. 6A, the individual rib segments forming the representative vertical rib 64 and the individual rib segments forming the representative horizontal rib 66 can be seen as being at right angles with adjoining rib segments. This may be considered as representing the undeformed configuration. In comparing FIG. 6A to FIG. 6B, it can be seen that the individual rib segments forming the representative vertical rib 64 now form angles with adjoining rib segments that are greater than 90 degrees. The individual rib segments forming the representative horizontal rib 66 now form angles with adjoining rib segments that are greater than 90 degrees, although in some instances, the individual rib segments forming the representative vertical rib 64 may form angles that are greater than the angles formed between the individual rib segments forming the representative horizontal rib 66. The auxetic structure 60 may be formed from any of a variety of known polymers, including polymers known in medical applications. Providing the polymeric tubular body 12 with the auxetic structure 60 will permit the polymeric tubular body 12 to increase in size radially in response to stretching the polymeric tubular body 12 axially.

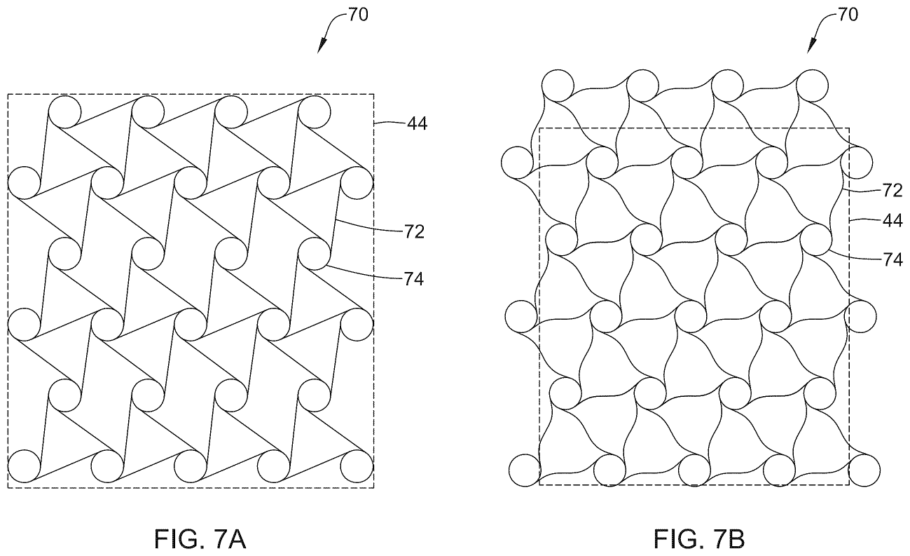

FIG. 7A is a schematic view of an example auxetic structure 70 shown in an undeformed or native configuration while FIG. 7B is a schematic view of the example auxetic structure 70 shown in a deformed configuration in which the auxetic structure 70 has increased in diameter as a result of being stretched axially. The auxetic structure 70 may be considered as being an example of a chiral auxetic structure. A chiral auxetic structure may be formed by connecting straight ligaments or ribs between central nodes, which may be circles or other geometric forms. Auxetic effects are achieved by the straight ligaments wrapping or unwrapping relative to the central nodes in response to applied forces. A difference between the undeformed configuration and the deformed configuration may be seen by comparing a dashed box 44 that in FIG. 7A, surrounds the auxetic structure 70, while in FIG. 7B, the auxetic structure 70 is wider and taller than the auxetic structure 70 as shown in FIG. 7A.

As shown, the auxetic structure 70 includes straight ligaments 72 extending between central nodes 74. In comparing FIG. 7A and FIG. 7B, it can be seen that in FIG. 7B, the individual straight ligaments 72 have partially unwrapped from being wrapped around the central nodes 74 to which they are attached, allowing the auxetic structure 70 to increase in volume. The auxetic structure 70 may be formed from any of a variety of known polymers, including polymers known in medical applications. Providing the polymeric tubular body 12 with the auxetic structure 70 will permit the polymeric tubular body 12 to increase in size radially in response to stretching the polymeric tubular body 12 axially.

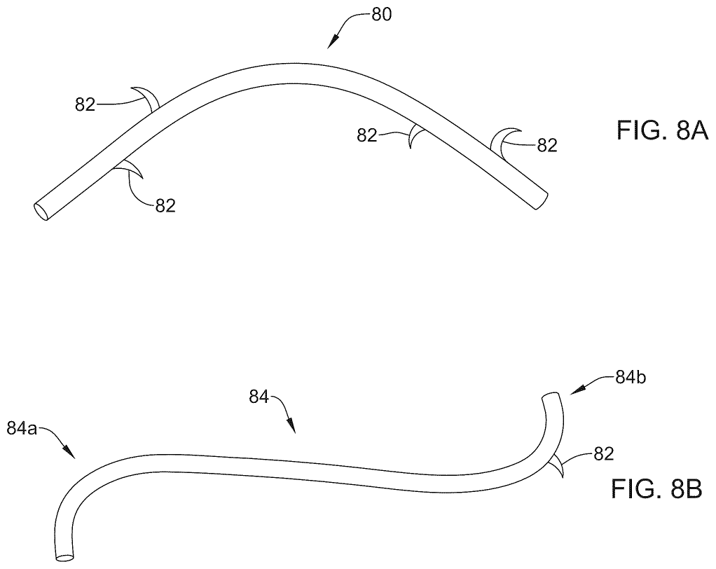

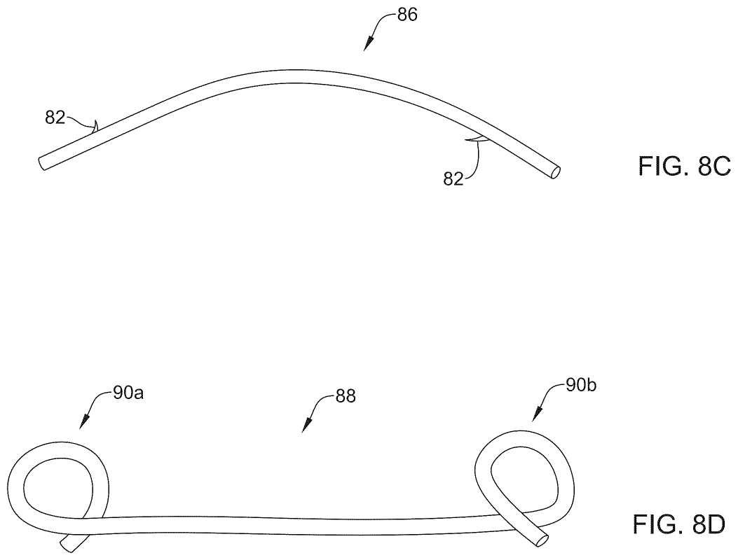

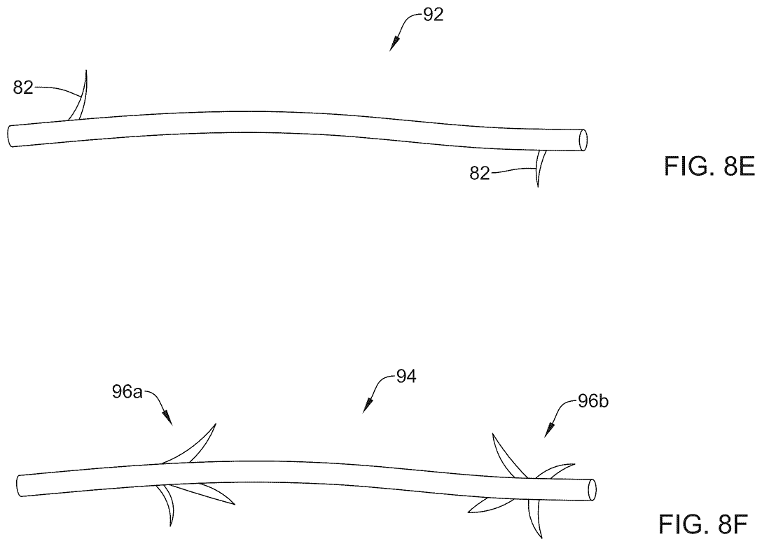

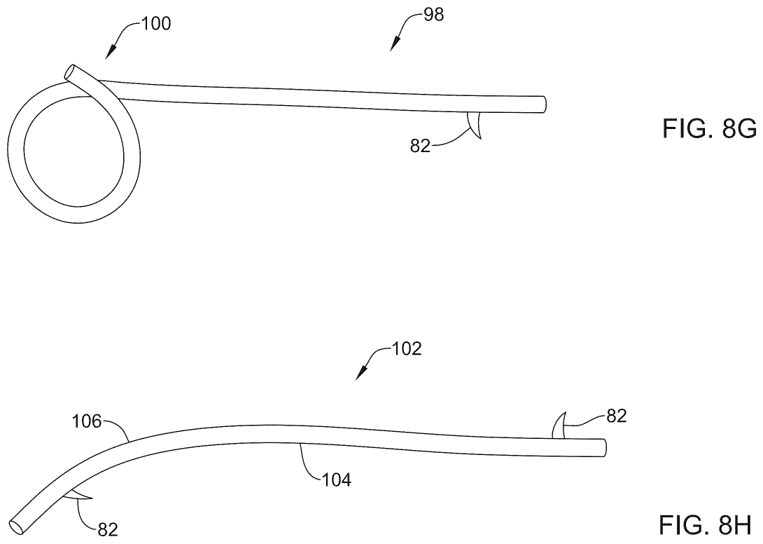

With brief reference back to FIGS. 1 and 2, the polymeric stent 12 may have a variety of different “remembered” shapes to which the polymeric stent 12 will return to once no longer constrained by a delivery device, for example. While the polymeric stent 12 is shown as being essentially cylindrical, it will be appreciated that in many instances a polymeric stent may include curves or bends, pigtails, barbs and other features. FIGS. 8A through 8H are schematic views of example polymeric stents that may each be considered as being examples of the polymeric stent 10 shown in FIGS. 1 and 2. FIG. 8A is a schematic view of a polymeric stent 80 that may be used in a pancreatic application. The polymeric stent 80 includes a curved shape, and includes a plurality of barbs 82. In some instances, the barbs 82 are formed by cutting a flap into the body of the polymeric stent 80. FIG. 8B is a schematic view of a polymeric stent 84 that may be referred to as an S-banded stent. The polymeric stent 84 includes a first bend 84a of about 90 degrees at a first end of the polymeric stent 84 and a second bend 84b of about 90 degrees at a second end of the polymeric stent 84. The first bend 84a and the second bend 84b may be seen as being in opposite directions, hence forming an elongated “S” shape. The polymeric stent 84 may include one or more barbs 82.

FIG. 8C is a schematic view of a polymeric stent 86. The polymeric stent 86 may be referred to as an Amsterdam stent. The polymeric stent 86 has a curved body, similar to the polymeric stent 80 (the Pancreatic stent), but has fewer barbs 82. FIG. 8D is a schematic view of a polymeric stent 88 that may be referred to as a double pigtail. The polymeric stent 88 includes a first pigtail 90a formed at a first end of the polymeric stent 88 and a second pigtail 90b formed at a second end of the polymeric stent 88. FIG. 8E is a schematic view of a polymeric stent 92 that may be considered as an example of a straight stent. The polymeric stent 92 includes a barb 82 at each end of the polymeric stent 92.

FIG. 8F is a schematic view of a polymeric stent 94. The polymeric stent 94 may be referred to as a tree type or pine tree stent. The polymeric stent 94 includes a first cluster of barbs 96a and a second cluster of barbs 96b that are spaced apart from the first cluster of barbs 96a. FIG. 8G is a schematic view of a polymeric stent 98. The polymeric stent 98 may be referred to as a single pigtail stent. The polymeric stent 98 includes a pigtail 100 formed at one end of the polymeric stent 98. In some instances, the polymeric stent 98 may include a barb 82 located at an end opposite that of the pigtail 100. FIG. 8H is a schematic view of a polymeric stent 102 that may be referred to as a standard stent. The polymeric stent 102 includes a straight body portion 104 and a curved body portion 106. The polymeric stent 102 may include a barb 82 near either end.

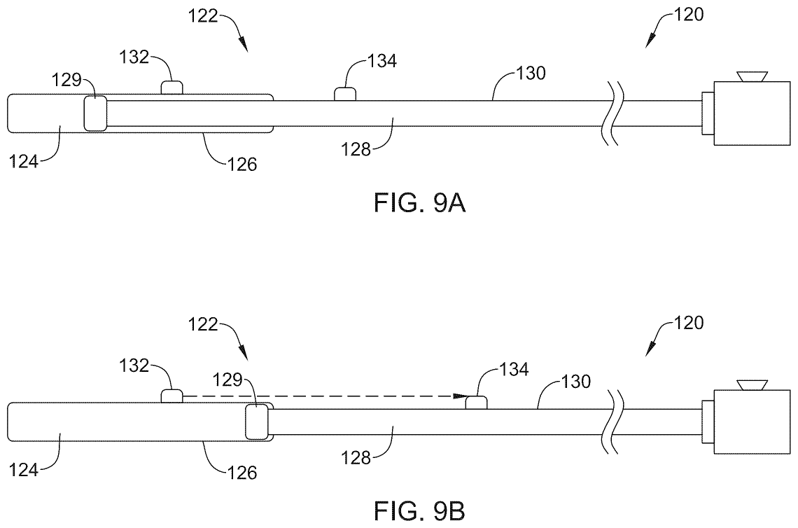

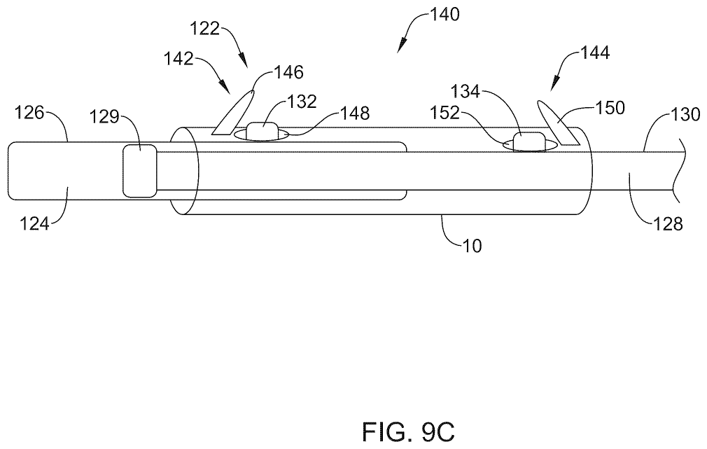

As shown, polymeric stents can have a variety of different shapes and configurations. These shapes and configurations may represent “remembered” shapes and configurations that the polymeric stent will regain once the polymeric stent is no longer constrained into a different configuration, such as a linear configuration, for delivery. FIGS. 9A through 9C are schematic views of an example delivery device 120 that may be used in delivering and subsequently expanding the polymeric stent 10. As seen in FIG. 9A, the delivery device 120 includes a tubular body 122 that is adapted to accommodate the polymeric stent 10 (not shown in this view) disposed over the tubular body 122. In some instances, the tubular body 122 may include an elongate outer member 124 having an outer surface 126 and an elongate inner member 128 having an outer surface 130. In some instances, the elongate inner member 128 is adapted to telescope or translate axially relative to the elongate outer member 124. In some instances, the elongate inner member 128 may include a distal stop 129. The distal stop 129 may be dimensioned to fit and translate within the elongate outer member 124, but to prevent the elongate inner member 128 from being pulled completely out of the elongate outer member 124.

In some instances, the delivery device 120 may include a distal stent engagement member 132 that is secured to the outer surface 126 of the elongate outer member 124, and hence may be considered as being secured relative to the tubular body 122. The delivery device 120 may include a proximal stent engagement member 134 that is secured to the outer surface 130 of the elongate inner member 128 and thus may be considered as being secured relative to the tubular body 122. The distal stent engagement member 132 may be adapted to engage with a corresponding distal engagement feature on the polymeric stent 10, and the proximal stent engagement member 134 may be adapted to engage with a corresponding proximal engagement feature on the polymeric stent 10. As an example, the distal stent engagement member 132 may include a distal protrusion that extends radially from the tubular body 122. The proximal stent engagement member 134 may include a proximal protrusion that extends radially from the tubular body 122. The elongate inner member 128 may be adapted to translate relative to the elongate outer member 124. In some instances, if the polymeric stent 10 is being delivered to a tight stricture, the distal stent engagement member 132 may not be necessary.

Accordingly, as shown in FIG. 9B, moving the elongate inner member 128 relative to the elongate outer member 124 can change the distance between the distal stent engagement member 132 and the proximal stent engagement member 134. As a result of the distal stent engagement member 132 being engaged with a distal engagement feature of the polymeric stent 10 and the proximal stent engagement member 134 being engaged with a proximal engagement feature of the polymeric stent 10, it will be appreciated that changing the distance between the distal stent engagement member 132 and the proximal stent engagement member 134 by translating the elongate inner member 128 within the elongate outer member 124 may change a length of the polymeric stent 10. In some instances, withdrawing the elongate inner member 128 proximally relative to the elongate outer member 124 causes the polymeric stent 10 to lengthen in response to an applied tensile force. As the polymeric stent 10 lengthens, as a result of the auxetic properties of the polymeric stent 10, the polymeric stent 10 will also increase in diameter.

FIG. 9C is a schematic view of an example assembly 140, which includes the polymeric stent 10 disposed over the tubular body 122. The polymeric stent 10 includes a distal engagement feature 142 that is formed within the distal region 14 of the polymeric stent 10 and a proximal engagement feature 144 that is formed within the proximal region 16 of the polymeric stent 10. As can be seen, the distal engagement feature 142 engages the distal stent engagement member 132 while the proximal engagement feature 144 engages the proximal stent engagement member 134. The polymeric stent 10 includes a distal barb 146 that may be formed by cutting into the polymeric stent 10 and bending the distal barb 146 radially outwardly, leaving an opening 148 formed in the polymeric stent 10. The polymeric stent 10 includes a proximal barb 150 that may be formed by cutting into the polymeric stent 10 and bending the proximal barb 150 radially, leaving an opening 152 formed in the polymeric stent 10. In some instances, the distal engagement feature 142 may include the distal barb 146 or the opening 148. In some instances, the distal engagement feature 142 may include a separately formed aperture. In some instances, the proximal engagement feature 144 may include the proximal barb 150 or the opening 152. In some instances, the proximal engagement feature 144 may include a separately formed aperture.

As can be seen, the polymeric stent 10 may be disposed over the tubular body 122, with the distal engagement feature 142 engaged with the distal stent engagement member 132 and the proximal engagement feature 144 engaged with the proximal stent engagement member 134. The assembly 140 may be advanced to a desired treatment location, whether that includes a trans-vascular approach or an endoscopic approach, for example. Once the polymeric stent 10 is in an appropriate position, the polymeric stent 10 may be deployed. Deploying the polymeric stent 10 includes withdrawing the elongate inner member 128 proximally relative to the elongate inner member 124, thereby increasing the distance between the distal stent engagement member 132 and the proximal stent engagement member 134. As a result, a corresponding increase in distance between the distal engagement feature 142 and the proximal engagement feature 144 occurs, and the polymeric stent 10 is stretched axially. As a result of being stretched axially, the polymeric stent 10 will increase in diameter.

In some instances, as the polymeric stent 10 continues to grow radially as a result of being stretched axially, the polymeric stent 10 will achieve a diameter at which the distal stent engagement member 132 no longer retains the distal engagement feature 142 and the proximal stent engagement member 134 no longer retains the proximal engagement feature 144. As a result, the polymeric stent 10 will no longer be held captive to the delivery device 120, and will be released. In some instances, if the distal stent engagement member 132 is still engaged with the distal engagement feature 142 and the proximal stent engagement member 134 is still engaged with the proximal engagement feature 144, moving the elongate inner member 128 distally relative to the elongate outer member 124 will cause the polymeric stent 10 to reduce in length, which will also cause the polymeric stent 10 to reduce in diameter in order to facilitate adjusting the location of the polymeric stent 10 before releasing the polymeric stent 10 from the delivery device 120.

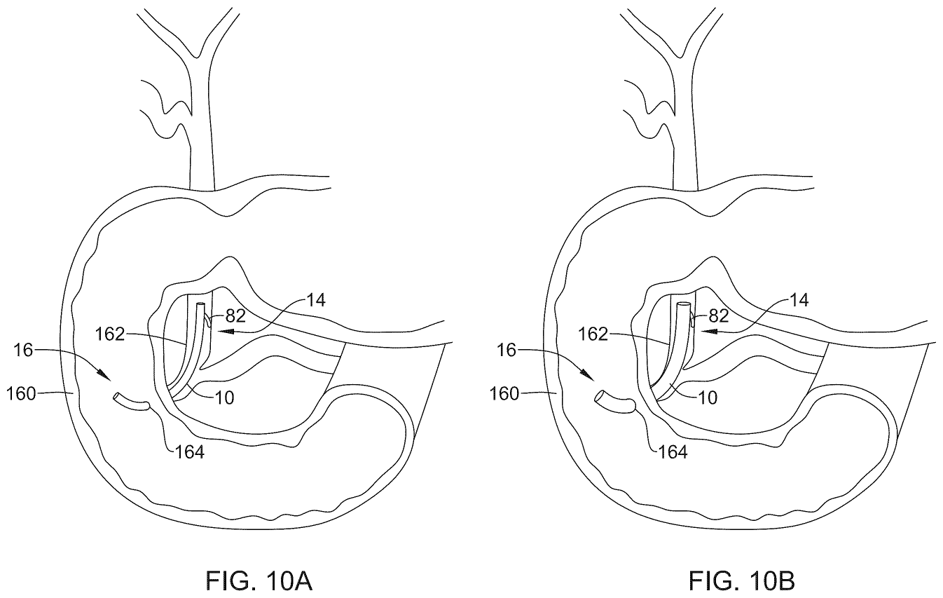

The delivery device 120 may be used to deliver the polymeric stent 10 to a variety of different locations in and near the biliary duct. FIGS. 10A and 10B are schematic views showing the duodenum 160. The bile duct 162 is fluidly coupled with the duodenum via the Papilla of vater 164. As seen in FIG. 10A, the polymeric stent 10 has been disposed within the bile duct 162, extending into the duodenum 160 a short distance through the Papilla of vater 164. At this point, the polymeric stent 10 has not yet been stretched axially, and thus may be considered as being disposed on the delivery device 120 (not shown). The polymeric stent 10 includes a barb 82 disposed at or near the distal region 14 of the polymeric stent 10. In FIG. 10B, the polymeric stent 10 has been lengthened and as a result has expanded radially. The polymeric stent 10 is able to be delivered in a low-profile configuration and can be expanded into a large profile configuration using the delivery device 120.

In some instances, if a tight stricture is being treated, the distal stent engagement member 132 may not be needed. The barb feature 82, which would be placed beyond the stricture, may have sufficient purchase due to the tightness of the bile duct 162 to act as a resistive element, and with the proximal stent engagement member 134 being withdrawn, may cause the polymeric stent 10 to expand both axially and radially. In some instances, anatomical sphincters may be used similarly to act as a temporary anchoring location to allow the polymeric stent 10 to be stretched and thus expanded.

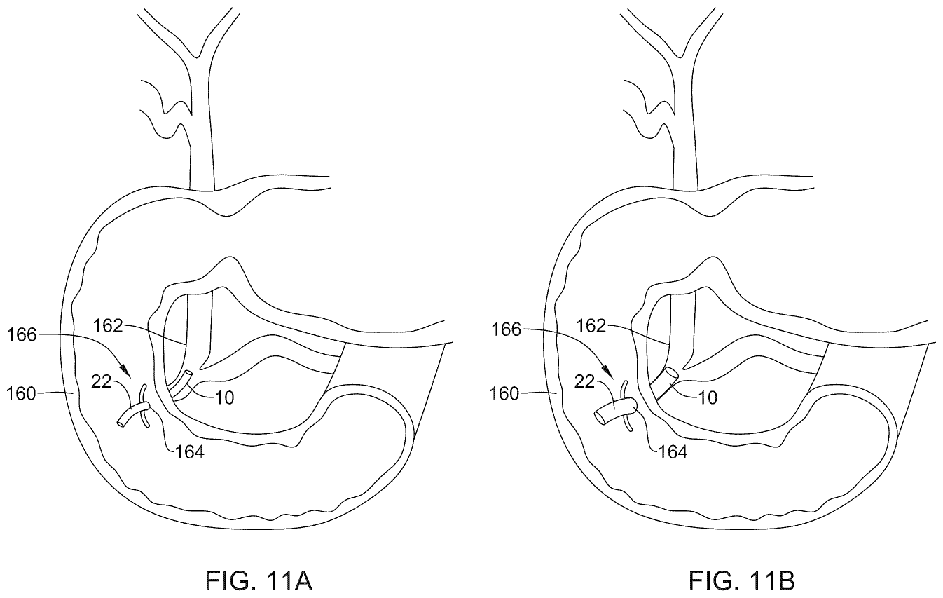

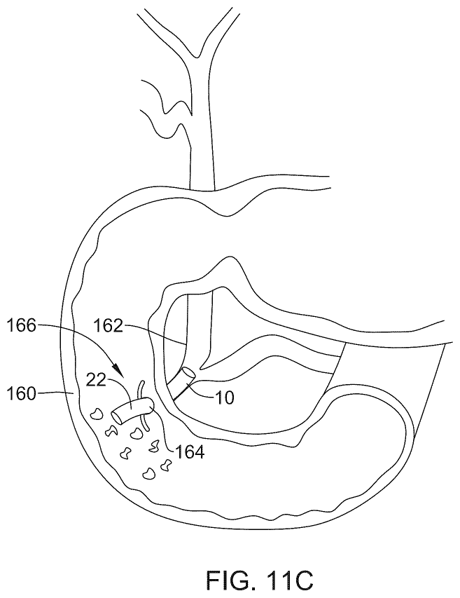

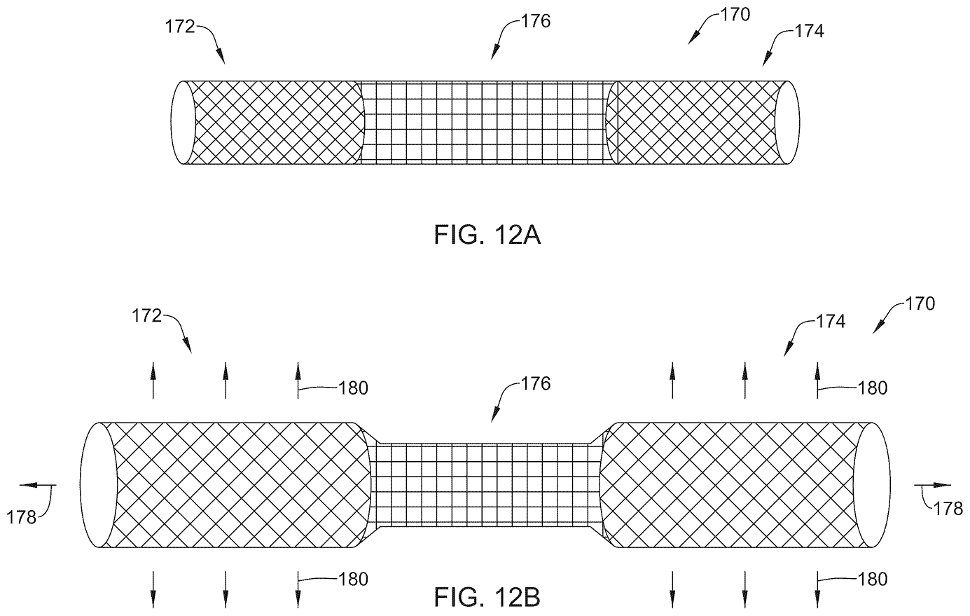

FIGS. 11A through 11C are schematic views that together show a laparoscopic approach. In FIG. 11A, the polymeric stent 10 has been advanced laparoscopically through the bile duct 162 to a position in which the distal end 22 of the polymeric stent 10 extends through the Papilla of vater 164 into the duodenum 160. The delivery device 120 is not illustrated. The distal end 22 of the polymeric stent 10 includes several barbs 166. Moving to FIG. 11B, the polymeric stent 10 has been expanded both laterally and radially, such as by withdrawing the elongate inner member 128 relative to the elongate outer member 124. As seen in FIG. 11C, biliary debris may be seen exiting the lumen 20 at the distal end 22 of the polymeric stent 10.

As discussed, the polymeric stent 10 is formed of an auxetic material, and thus is able to expand radially in response to being stretched axially. In some instances, a polymeric stent may include portions or segments having auxetic properties and other portions or segments having non-auxetic properties. FIG. 11A is a schematic view of an example polymeric stent 170. The example polymeric stent 170 includes a distal region 172, a proximal region 174 and an intermediate region 176. The polymeric stent 170 may be considered as having a relaxed configuration (as shown) in which the polymeric stent 170 has a uniform diameter. In some instances, the distal region 172 and the proximal region 174 may be formed of an auxetic material while the intermediate region 176 is formed of a non-auxetic material. Turning to FIG. 11B, it can be seen that the polymeric stent 170 has been stretched axially, applying a tensile force in a direction indicated by the arrows 178. As a result, the auxetic material within the distal region 172 and the proximal region 174 expands radially, in a direction indicated by arrows 180, thereby forming distal and proximal flares to protect against migration, for example. The non-auxetic material within the intermediate region 176 does not materially expand. In some instances, the non-auxetic material within the intermediate region 176 may actually decrease in diameter as a result of the applied tensile force.

The materials that can be used for the various components of the medical stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. An assembly, comprising:
a polymeric stent including:
a polymeric tubular body extending from a distal region to a proximal region, the polymeric tubular body adapted to have a negative Poisson's ratio;
a distal engagement feature formed within the distal region; and
a proximal engagement feature formed within the proximal region; and
a delivery device adapted to deliver the polymeric stent, the delivery device including:
a tubular body adapted to accommodate the polymeric stent disposed over the tubular body;
a distal stent engagement member secured relative to the tubular body, the distal stent engagement member adapted to releasably engage the distal engagement feature;
a proximal stent engagement member secured relative to the tubular body, the proximal stent engagement member adapted to releasably engage the proximal engagement feature;
wherein the tubular body is adapted to affect a change in a distance between the distal stent engagement member and the proximal stent engagement member, thereby changing a length of the polymeric stent.

2. The assembly of claim 1, wherein increasing the length of the polymeric stent results in the polymeric stent increasing in diameter.

3. The assembly of claim 2, wherein subsequently decreasing the length of the polymeric stent after increasing the length of the polymeric stent results in the polymeric stent decreasing in diameter.

4. The assembly of claim 1, wherein the distal stent engagement member and the proximal engagement member are each adapted to allow the polymeric stent to disengage from the distal stent engagement member and the proximal engagement member once the proximal stent reaches a predetermined length.

5. The assembly of claim 1, wherein:
the distal stent engagement member comprises a distal protrusion extending above the outer surface of the tubular body; and
the proximal stent engagement member comprises a proximal protrusion extending above the outer surface of the tubular body.

6. The assembly of claim 5, wherein:
the distal engagement feature comprises a distal aperture formed within the distal region, the distal aperture adapted to releasably engage the distal stent engagement member; and
the proximal engagement feature comprises a proximal aperture formed within the proximal region, the proximal aperture adapted to releasably engage the proximal stent engagement member.

7. The assembly of claim 5, wherein
the distal engagement feature comprises a distal barb formed within the distal region, the distal barb adapted to releasably engage the distal stent barb member; and
the proximal engagement feature comprises a proximal aperture formed within the proximal region, the proximal barb adapted to releasably engage the proximal stent engagement member.

8. The assembly of claim 1, wherein the tubular body comprises:
an elongate outer member including an outer surface; and
an elongate inner member, the elongate inner member including an outer surface;
wherein:
the distal stent engagement member is secured to the outer surface of the elongate outer member;
the proximal stent engagement member secured to the outer surface of the elongate inner member.

9. The assembly of claim 8, wherein the elongate inner member is adapted to translate within the elongate outer member, thereby changing the distance between the distal stent engagement member and the proximal stent engagement member.

10. The assembly of claim 1, wherein the polymeric tubular body comprises a rotating rectangles auxetic structure.

11. The assembly of claim 1, wherein the polymeric tubular body comprises a re-entrant auxetic structure.

12. The assembly of claim 1, wherein the polymeric tubular body comprises a chiral auxetic structure.

13. A delivery device for delivering a polymeric stent that includes a polymeric tubular body adapted to grow radially in response to being stretched axially, the polymeric stent including a first engagement feature and a second engagement feature, the delivery device comprising:
a telescoping body including an elongate outer member and an elongate inner member, the elongate inner member adapted to translate within the elongate outer member;
a first stent engagement member secured relative to the elongate outer member, the first stent engagement member adapted to releasably engage the first engagement feature;
a second stent engagement member secured relative to the elongate inner member, the second stent engagement member adapted to releasably engage the second engagement feature;
wherein translating the elongate inner member in a first direction relative to the elongate outer member causes a distance between the first stent engagement member and the second stent engagement member to increase, thereby stretching the polymeric stent and causing the polymeric stent to increase in diameter.

14. The delivery device of claim 13, wherein the distal stent engagement member and the proximal engagement member are each adapted to allow the polymeric stent to disengage from the distal stent engagement member and the proximal engagement member once the proximal stent reaches a predetermined length.

15. The delivery device of claim 13, wherein:
the distal stent engagement member comprises a distal protrusion extending radially away from the elongate outer member; and
the proximal stent engagement member comprises a proximal protrusion extending radially away from the elongate inner member.

16. The delivery device of claim 13, wherein the elongate inner member comprises a distal stop disposed at a distal end of the elongate inner member, the distal stop adapted to limit how far the elongate inner member can be withdrawn from the elongate outer member.

17. The delivery device of claim 13, wherein:
the polymeric stent comprises a first barb and associated hollow as the first engagement feature and a second barb and associated hollow as the second engagement feature;
the first stent engagement member is adapted to releasably engage the first barb and associated hollow; and
the second stent engagement member is adapted to releasably engage the second barb and associated hollow.

18. A polymeric stent adapted for placement within a biliary duct, the polymeric stent comprising:
a polymeric tubular body extending from a distal region to a proximal region;
the polymeric tubular body having a delivery configuration with a first diameter and a first length;
the polymeric tubular body having a deployed configuration with a second diameter greater than the first diameter and a second length greater than the first length;
wherein the polymeric tubular body is adapted to increase in diameter from the first diameter towards the second diameter in response to the polymeric tubular body being stretched axially from the first length towards the second length.

19. The polymeric stent of claim 18, wherein the polymeric tubular body comprises an auxetic structure.

20. The polymeric stent of claim 19, wherein the auxetic structure comprises one of a rotating rectangles auxetic structure, a re-entrant auxetic structure or a chiral auxetic structure.
